(19) United States
(12) Patent Application Publication
(10) Pub. No.: US 2005/0148854 A1
(43) Pub. Date: Jul. 7, 2005

(54) Diagnosis supporting device

(75) Inventors: Eiichi Ito, Tokyo (JP); Kyojiro Nambu, Tochigi (JP)

Correspondence Address: GREENBLUM & BERNSTEIN, P.L.C., 1950 ROLAND CLARKE PLACE, RESTON, VA, 20191, US

(73) Assignee: PENTAX Corporation, Tokyo (JP)

(21) Appl. No.: 11/016,913

(22) Filed: Dec. 21, 2004

(30) Foreign Application Priority Data
Dec. 22, 2003 (JP) P2003-424646
Dec. 1, 2004 (JP) P2004-349139

(51) Int. Cl. A61B 5/05

(52) U.S. Cl. 600/407; 600/410

(57) ABSTRACT

Disclosed is a diagnosis supporting device that includes an endoscope device that takes an image of an internal structure of a subject by forming the image on an image sensor through an optical system, an image composing device that superimposes a perspective image of a predetermined area of the subject that is created based on sectional images obtained by a tomography scanner over an endoscopic image of the predetermined area taken by the endoscope device, a displaying device that displays the image composed by the image composing device, a first shifting mechanism that relatively shifts the position of the image formed by the optical system of the endoscope device and the position of the image sensor, and a second shifting mechanism that shifts the display area of the perspective image corresponding to the change of the image taking area by the first shifting mechanism.

### BACKGROUND OF THE INVENTION

The present invention relates to a diagnosis supporting device for displaying a composite image, which is created by superimposing a perspective image of a body captured by a tomography scanner such as a CT scanner or an MRI machine over an endoscopic image inside a body taken by a video endoscope device, on a monitor screen to support diagnosis by an operator.

Devices that display the composite images created by superimposing perspective images captured by a tomography scanner over endoscopic images on monitor screens during an operation are previously known. For example, Japanese unexamined patent publication No. 2002-102249 discloses an operation navigating device. The device creates three-dimensional information of a subject by modifying data of the subject measured by a tomography scanner before an operation with a predicted deformation due to the operation, and stores the three-dimensional information into a database. The device reads the three-dimensional information that is similar to a shape of the subject measured during the operation out of the database to create a perspective image (a data image) and displays the perspective image superimposed over an endoscopic image taken by a rigid endoscope on a monitor.

Further, Japanese unexamined patent publication No. 2002-224138 discloses a device that adjusts a positional relationship between a perspective image created based on data of a subject measured by a tomography scanner before an operation and an endoscopic image taken by a rigid endoscope. The device stores types and individual differences of tools such as a rigid endoscope in a database. When the images are overlapped, the device extracts the data of the tools in active use from the database and adjusts the positional relationship between the images according to the extracted data.

On the other hand, devices that superimpose a cursor showing an area observed by an endoscope or a position of a surgical instrument over a perspective image are previously known. For example, Japanese unexamined patent publication No. 2001-198141 disclose a rigid endoscope that has a CCD moving mechanism in a connected television camera so that the observation area can be shifted without moving the tip end of the endoscope. The operation area observing system disclosed in this publication superimposes a cursor showing an observation area of the rigid endoscope over a perspective image created based on the data of a subject measured by a CT scanner, an MRI machine or the like before an operation.

Further, Japanese unexamined patent publication No. 2001-293006 discloses a device that displays a position of a surgical instrument on a perspective image. The device disclosed in this publication extracts a sectional image from stored sectional images measured by a CT scanner or an MRI machine before an operation based on three-dimensional position/attitude information of a subject and superimposes the position and attitude of the surgical instrument over the extracted sectional image based on three-dimensional position/attitude information of the surgical instrument.

Still further, Japanese unexamined patent publication No. 2002-17751 discloses an operation navigating device that extracts a sectional image from stored sectional images measured by a CT scanner or an MRI machine before an operation based on three-dimensional position/attitude information of a subject and superimposes the position and attitude of the surgical instrument over the extracted sectional image based on three-dimensional position/attitude information of the surgical instrument. The device measures a distance between the subject and the surgical instrument and changes a magnification of the displayed image according to the distance information.

However, the operation navigating devices disclosed in Japanese unexamined patent publications No. 2002-102249 and No. 2002-224138 have to move the endoscope devices to change the observation area. Therefore, the position of the endoscope device must be reset even if the observation area will be slightly moved, which complicates a handling.

On the other hand, the devices disclosed in Japanese unexamined patent publications No. 2001-198141, No. 2001-293006 and No. 2002-17751 only superimpose the observation area of the endoscope device or the position of the surgical instrument over the perspective image. Therefore, the positional relationship between the information (a shape of a body cavity wall or the like) represented by the endoscopic image and the information (a position of a blood vessel or the like) represented by the perspective image cannot be directly grasped when an endoscope device is used.

### SUMMARY OF THE INVENTION

It is therefore an object of the present invention to provide an improved diagnosis supporting device that is capable of changing an observation area without changing a position of an endoscope device and is capable of grasping a positional relationship between information represented by an endoscopic image and information represented by a perspective image easily by an operator.

A diagnosis supporting device of the present invention includes an endoscope device that takes an image of an internal structure of a subject by forming the image on an image sensor through an optical system, a holding mechanism that holds the endoscope device so that the endoscope device can be fixed with respect to the subject, an image composing device that superimposes a perspective image of a predetermined area of the subject that is created based on sectional images obtained by a tomography scanner over an endoscopic image of the predetermined area taken by the endoscope device, a displaying device that displays the image composed by the image composing device, a first shifting mechanism that relatively shifts the position of the image formed by the optical system of the endoscope device and the position of the image sensor, and a second shifting mechanism that shifts the display area of the perspective image corresponding to the change of the image taking area by the first shifting mechanism. The endoscopic image shifted by the first shifting mechanism and the perspective image shifted by the second shifting mechanism are composed by the image composing device, and the composed image is displayed on the displaying device.

The tomography scanner may be a CT scanner or an MRI machine. Further, when the optical system of the endoscope device includes a Pechan prism, the first shift mechanism may consist of the Pechan prism and a prism moving mechanism that moves the prism in a plane perpendicular to an optical axis of the optical system.

Still further, the endoscope device preferably includes an objective optical system that forms an image of the subject, a first re-imaging optical system that re-images a predetermined area of the image formed by the objective optical system, a first image sensor that takes the image formed by the first re-imaging optical system, a second re-imaging optical system that enlarges and re-images a part of the predetermined area of the image formed by the objective optical system, and a second image sensor that takes the image formed by the second re-imaging optical system. In such a case, the image composing device creates a first composite image by composing a first endoscopic image taken by the first image sensor with a perspective image of the corresponding area and creates a second composite image by composing a second endoscopic image taken by the second image sensor with a perspective image of the corresponding area. Then, the first shifting mechanism shifts the relative position of the image formed by the second re-imaging optical system and the second image sensor, and the second shifting mechanism shifts the perspective image that will constitute the second composite image. The displaying device preferably includes a first monitor for displaying the first composite image and a second monitor for displaying the second composite image.

According to the present invention, the observation area can be changed by the first shifting mechanism without moving the position of the endoscope device, and the display area of the perspective image is changed by the second shifting mechanism in response to the shift by the first shifting mechanism. Since the areas of the both images that are superimposed can be coincident with each other, an operator can easily grasp the positional relationship between information represented by the endoscopic image and information represented by the perspective image.

### DESCRIPTION OF THE ACCOMPANYING DRAWINGS

FIG. 1 is a block diagram showing an outline of a diagnosis supporting device according to an embodiment of the present invention;

FIG. 2A shows a sample image displayed on a first monitor included in the diagnosis supporting device of FIG. 1;

FIG. 2B shows a sample image displayed on a second monitor included in the diagnosis supporting device of FIG. 1;

FIG. 3 shows an inner construction including an optical system of an endoscope device in the diagnosis supporting device of FIG. 1;

FIG. 4 is an enlarged perspective view of a Pechan prism used in the endoscope device of FIG. 3;

FIG. 5A is a plan view of an endoscope marker attached to the endoscope device included in the diagnosis supporting device of FIG. 1;

FIG. 5B is a side view of the endoscope marker;

FIG. 6 shows a holding mechanism included in the diagnosis supporting device of FIG. 1;

FIG. 7A is a plan view of a position measuring device included in the diagnosis supporting device of FIG. 1;

FIG. 7B is a side view of the position measuring device;

FIG. 7C is a front view of the position measuring device;

FIG. 8A is a perspective view showing a coordinate system of the position measuring device;

FIG. 8B is a side view showing the coordinate system of the position measuring device;

FIG. 9 shows a perspective image reference position marker;

FIG. 10 shows a coordinate system of the tomography scanner of the embodiment;

FIG. 11 is a block diagram showing an outline of an image composing device included in the diagnosis supporting device of FIG. 1;

FIG. 12 is a flowchart showing image composing process executed by the image composing device of FIG. 11;

FIG. 13 shows coordinate transformation from a cylindrical coordinate to a local coordinate; and

FIG. 14 shows an imaging condition in the endoscope included in the diagnosis supporting device of FIG. 1.

### DESCRIPTION OF THE EMBODIMENTS

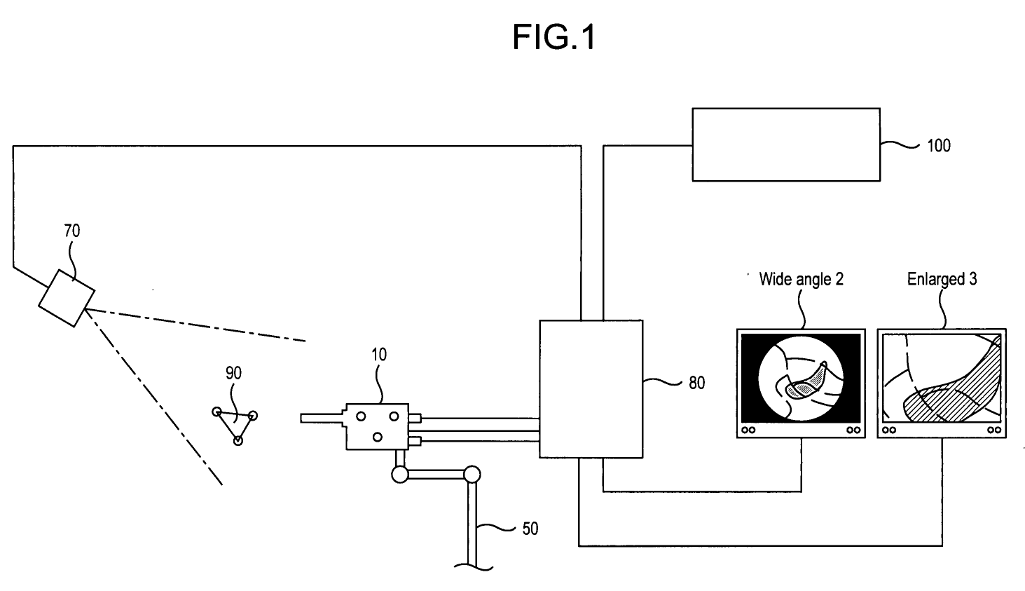

An embodiment of the present invention will be described hereinafter with reference to the drawings. FIG. 1 is a block diagram showing an outline of a diagnosis supporting device of the embodiment.

As shown in FIG. 1, the diagnosis supporting device of the embodiment includes an endoscope device 10 that takes an image of an internal structure of a subject to be diagnosed formed on an image sensor through an optical system, a holding mechanism 50 that holds the endoscope device 10 with respect to the subject, a position measuring device 70 that measures the position of the endoscope device 10, an image composing device 80 that superimposes a perspective image of a predetermined portion of the subject over an endoscopic image of the predetermined portion taken by the endoscope device 10, and first and second monitors 2 and 3 as displaying devices that display the superimposed images formed by the image composing device 80. The perspective image is created based on sectional images obtained by a tomography device 100 such as a CT scanner or a MRI machine. In addition, the reference number 90 denotes a perspective image reference position marker that is attached to the subject so that the position of the subject can be measured by the position measuring device 70 when the tomography scanner 100 takes sectional images.

The perspective image here means a two-dimensional image that is a sight of internal structures from a given viewpoint calculated from a three-dimensional data obtained by reconstructing section images of a subject captured by a tomography scanner. The two-dimensional image can be formed by a general method used in computer graphics and an imaging diagnostic device. For example, there are surface lettering that extracts a predetermined surface (a boundary between air and material other than air, for example) of a three-dimensional subject, volume lettering that virtually sees through a subject based on numerical values representing physical characteristics of the respective points in the subject, and wire-frame lettering that shows general shape of an important portion (blood vessel, for example) selected from internal structures of a subject as a three-dimensional line drawing.

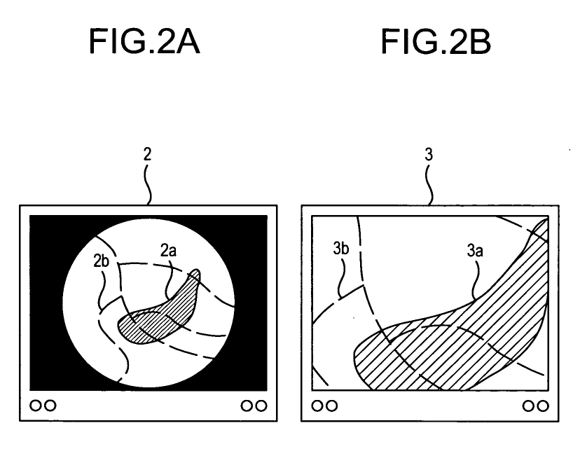

The endoscope device 10 has two image-taking systems whose taking areas have different sizes as described below. As shown in FIG. 2A and FIG. 2B, the first monitor 2 displays a wide-angle composite image that is formed by superimposing the endoscopic image taken by one image-taking system with the corresponding perspective image, and the second monitor 3 displays an enlarged composite image that is formed by superimposing the endoscopic image taken by the other image-taking system with the corresponding perspective image. In FIGS. 2A and 2B, structures in the body 2a and 3a included in the endoscopic images and simplified images of structures such as blood vessels 2b and 3b in the internal organ included in the perspective image are displayed in superimposed fashion.

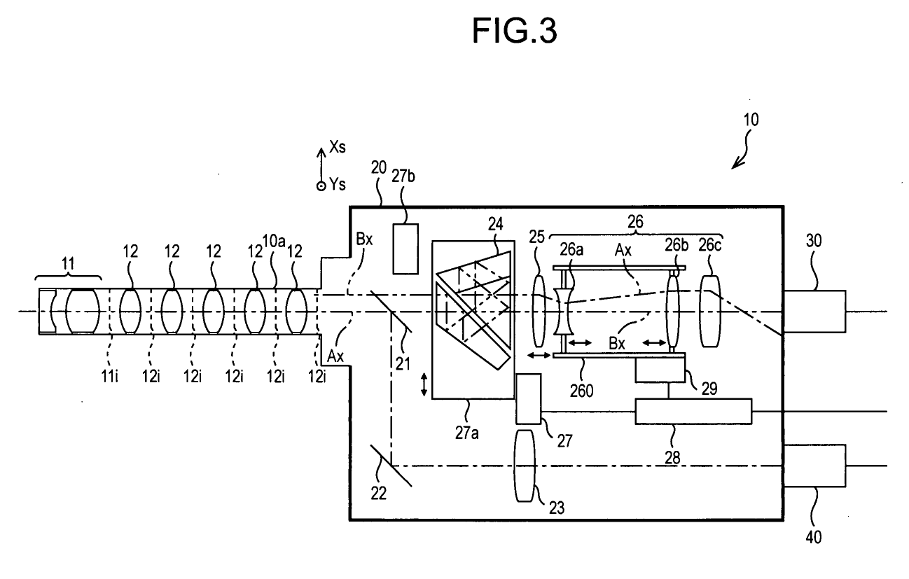

The endoscope device 10 has, as shown in FIG. 3, a rigid endoscope 10a whose tip end can be inserted into a body cavity through a trocar that is set at abdominal wall of a patient as a subject, an image separating device 20 to which the rigid endoscope 10a is attached, and, first and second CCD cameras 40 and 30 that take images relayed by optical systems mounted in the image separating device 20.

The rigid endoscope 10a is provided with an objective optical system that forms an image inside the body cavity and relays it, and a light guide (not shown) that guides illumination light from a light source device (not shown) to the tip portion to illuminate the inside of the body cavity. The objective optical system and the light guide are installed in a linear tube. The objective optical system consists of an objective lens group 11 and a plurality of relay lenses 12. The objective lens group 11 is a retro-focus type lens that can form an image of a wide viewing area (the angle of view is larger than 120 degrees, for example). The image inside the body cavity is formed on an imaging plane 11i by the objective lens group 11. The image on the imaging plane 11i is sequentially relayed by the respective relay lenses 12 and the image is formed on the imaging plane 12i of the last relay lens 12.

The image separating device 20 includes a half mirror 21, a folding mirror 22, a first re-imaging lens (a first re-imaging optical system) 23, a Pechan prism 24, a focusing lens 25, and a second re-imaging lens (a second re-imaging optical system) 26 that includes first, second and third lens groups 26a, 26b and 26c. The half mirror 21 is arranged on the optical path of the light flux from the objective optical system in the rigid endoscope 10a to reflect a part of the light flux and to permit transmission of the remainder of the light flux. The light flux reflected by the half mirror 21 is reflected by the folding mirror 22 and re-forms an image of a predetermined area of the subject on an image-taking surface of the first CCD camera 40 through the first re-imaging lens 23. The optical system from the objective optical system to the first re-imaging lens 23 corresponds to a first image taking optical system. The first CCD camera 40 corresponds to a first image sensor that takes an image formed by the first image taking optical system.

On the other hand, the light flux transmitted through the half mirror 21 is reflected in the Pechan prism 24 and re-forms an enlarged image of a part in the predetermined area of the subject on an image-taking surface of the second CCD camera 30 through the focusing lens 25 and the second re-imaging lens 26. The optical system from the objective optical system to the second re-imaging lens 26 corresponds to a second image taking optical system. The second CCD camera 30 corresponds to a second image sensor that takes an image formed by the second image taking optical system.

The first and second CCD cameras 40 and 30 are cameras for taking dynamic picture images that use ordinary solid-state image sensing devices (CCD). The cameras convert the light flux incident on the image taking surfaces into electric signals as image signals and output first and second image signals, respectively, to the image composing device 80.

In the first image taking optical system, an optical axis Ax of the objective optical system is bended by the half mirror 21 and is further cranked by the folding mirror 22. The cranked optical axis passes through the center of the first re-imaging lens 23 and goes through the center of the image taking surface of the first CCD camera 40 vertically.

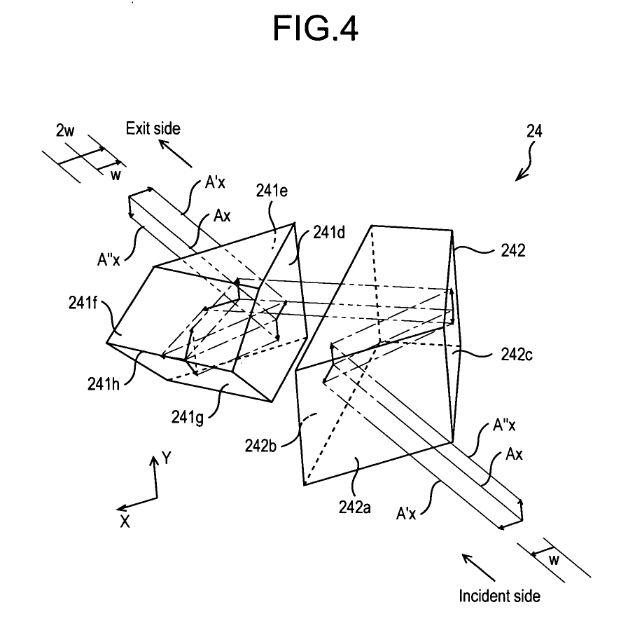

On the other hand, the Pechan prism 24 in the second image taking optical system functions as an optical axis shifting element and an image inverting optical system. The Pechan prism 24 is mounted on an XY stage 27a that is driven by a moving mechanism 27 so that the prism can move in an X-direction and a Y-direction in a plane perpendicular to the optical axis Ax of the objective optical system. FIG. 4 is a perspective view of the Pechan prism 24. As shown in FIG. 3 and FIG. 4, the Pechan prism 24 includes a roof prism 241 that has a shape equivalent to a triangle pole with roof surfaces 241f and 241g that are projected from one side surface of the triangle pole (the ridge line of the roof surfaces is parallel to the bottom surface of the triangle pole) and an auxiliary prism 242 having a square pole shape that is arranged so that a side surface 242b is close to and parallel to a side surface 241d of the roof prism 241 that is different from the surface to which the roof surfaces 241f and 241g are formed.

In the second image taking optical system, the optical axis Ax of the objective optical system vertically intersects the side surface 242a of the auxiliary prism 242 and is bended twice by the two side surfaces 242b and 242c that are adjacent to the side surface 242a. The optical axis vertically intersects the side surface 242b of the auxiliary prism 242 and the side surface 241d of the roof prism 241. Then, the optical axis Ax is bended by the side surface 241e, the roof surfaces 241f, 241g, and the side surface 241d of the roof prism 241 and vertically intersects the side surface 241e (the optical axis after exiting the roof prism 241 is parallel to that before entering the auxiliary prism 242). The position of the Pechan prism 24 where the optical axis Ax before entering the Pechan prism 24 is coaxial to that after exiting the Pechan prism 24 is referred to as an initial position in the following description. When the Pechan prism 24 is set at the initial position, the optical axis Ax passes through the centers of the focusing lens 25 and the second re-imaging lens 26, and goes through the center of the image taking surface of the second CCD camera 30 vertically.

The first and second lens groups 26a and 26b, which are variable power lenses in the second re-imaging lens 26, are supported by a zoom barrel 260 so that the lens groups can be moved along the optical axis. The third lens group 26c is fixed. The first and second lens groups 26a and 26b have cam followers connected to cam grooves formed on a cam ring (not shown) that constitutes the zoom barrel 260. When the cam ring rotates about the optical axis, the first and second lens groups 26a and 26b moves in the optical axis direction. Thereby, the magnification of the second re-imaging lens 26 can be adjusted. A zooming actuator (not shown) that employs a DC servomotor, a stepping motor or the like can be used as a drive source for the cam ring.

With the above described arrangement, the light flux transmitted through the half mirror 21 transmits the Pechan prism 24, the focusing lens 25 and the second re-imaging lens 26 in order, and is incident on the image taking surface of the second CCD camera 30. At this time, the Pechan prism 24 inverts the image formed by the objective optical system (the objective lens group 11 and the relay lenses 12) in the rigid endoscope 10a and the second re-imaging lens 26 re-forms an enlarged image, which is a part of the image formed by the objective optical system and inverted by the Pechan prism 24, on the image taking surface of the second CCD camera 30.

In addition, the moving mechanism 27 for moving the Pechan prism 24 in the XY plane is provided with a driving mechanism for the X table and a driving mechanism for the Y table. Each of the driving mechanisms includes a driving actuator such as a DC servomotor, a stepping motor or the like and a gear mechanism to transmit driving power of the actuator to the stage. As a result, the respective stages can be independently moved. The moving mechanism 27 and the Pechan prism 24 constitute the first shifting mechanism (the shifting device) that shifts the relative position of the image formed by the optical system of the endoscope device with respect to the image sensor. Further, the moving mechanism 27 is connected to a control device (not shown) having a joystick that can tilt in cross directions. When an operator controls the joystick of the control device, a signal corresponding to the tilting amount and the tilting direction of the joystick is transmitted to the moving mechanism 27. Since the moving mechanism 27 drives the X stage and/or the Y stage corresponding to the tilting amount and the tilting direction of the joystick, the XY stage 27a moves the Pechan prism 24 in the XY plane. The control device may have a track ball instead of the joystick. In such a case, the control device outputs a signal corresponding to a rotation amount and a rotation direction of the track ball rotated by an operator. Further, the control device may have a first lever for the movement in the X direction and a second lever for the movement in the Y direction. In such a case, the control device outputs a signal corresponding to the tilting amounts of the respective levers. The coordinate system (X_{S}, Y_{S}) of the XY stage is defined as shown in FIG. 3.

FIG. 4 shows the shift of the optical axis Ax of the objective optical system by the function of the Pechan prism 24. As shown in FIG. 4, when the optical axis Ax at the incident side is moved by a distance w in a positive direction (leftward in FIG. 4) of the X direction from the initial position (the moved optical axis is Ax′), the optical axis Ax′ at the exit side moves by a distance w in a negative direction of the X direction with respect to the optical axis Ax before the movement. This is equivalent to move the Pechan prism 24 with respect to the fixed optical axis Ax of the objective optical system by a distance w in the negative direction (rightward in FIG. 4) of the X direction. In such a case, the optical axis Ax′ of the objective optical system at the exit side is shifted by a distance 2w in the negative direction of the X direction with respect to the optical axis Ax′ at the incident side of the Pechan prism 24. On the contrary, when the Pechan prism 24 is moved in the positive direction of the X direction, the optical axis Ax of the objective optical system is shifted by the twofold distance of the moving amount of the Pechan prism 24 in the positive direction of the X-direction. Further, when the Pechan prism 24 is moved in the Y direction (an up-and-down direction in FIG. 4), the optical axis Ax″ of the objective optical system at the exit side of the Pechan prism 24 is shifted by the twofold distance of the moving amount of the Pechan prism 24 in the same direction of the movement of the Pechan prism 24 with respect to the optical axis Ax″ at the incident side of the Pechan prism 24.

As described above, when the Pechan prism 24 is shifted in the XY plane, the optical axis Ax of the objective optical system at the exit side of the Pechan prism 24 shifts from an optical axis Bx of the second re-imaging lens 26. FIG. 3 also shows this condition. At the initial position of the Pechan prism 24, the light that travels on the optical axis of the objective optical system also travels on the optical axis Bx of the second re-imaging lens 26 after exiting from the Pechan prism 24 and is incident on the center of the image taking surface of the second CCD camera 30. When the Pechan prism 24 is moved in the XY plane as shown in FIG. 4 from the initial position, the optical axis Ax at the exit side of the Pechan prism 24 is shifted from the optical axis Bx of the second re-imaging lens 26. Accordingly, the light that travels on the optical axis Ax of the objective optical system is incident on the second re-imaging lens 26 along a path shifted from the optical axis Bx and is incident on the image taking surface of the second CCD camera 30 at a point shifted from the center of the image taking surface. As a result, the image taking area of the second CCD camera 30 is shifted.

In addition, since the objective optical system of the rigid endoscope 10a has the objective lens group 11 that has a wide view angle and the relay lenses 12 that relay the image formed by the objective lens group 11, the objective optical system has large curvature of field. Therefore, when the Pechan prism 24 is moved in the XY plane to shift the image formed by the objective optical system in the X, Y directions with respect to a pupil of the second re-imaging lens 26, the image plane moves along the optical axis Bx of the second re-imaging lens 26 with respect to a point being conjugate to the image taking surface of the second CCD camera 30. As a result, the second CCD camera 30 may go out of focus. In the image separating device 20 of the embodiment, a focusing control circuit (not shown) drives the focusing actuator in synchronization with the moving mechanism 27 corresponding to the shifting amount of the optical axis Ax of the objective optical system with respect to the optical axis Bx of the second re-imaging lens 26 so that the image plane is coincident with the image taking surface of the second CCD camera 30.

Still further, the image separating device 20 is provided with a position detector 29 to detect the positions of the first and second lens groups 26a and 26b of the second re-imaging lens 26 that are moved by a zooming actuator (not shown) along the optical axis. The position detector 29 informs the detected position to the image composing device 80.

Specifically, the position detector 29 has an encoder that detects the rotating position of the cam ring of the zoom barrel 260 that holds the first and second lens groups 26a and 26b. The position detector 29 informs the detected information representing the rotation position of the cam ring as position information (the zoom position information) of the first and second lens groups 26a and 26b to the image composing device 80.

Since the endoscope device 10 can move the observation area of the second CCD camera 30 by the optical shifting mechanism, the observation area can be changed without moving the endoscope device 10 after the endoscope device 10 is fixed.

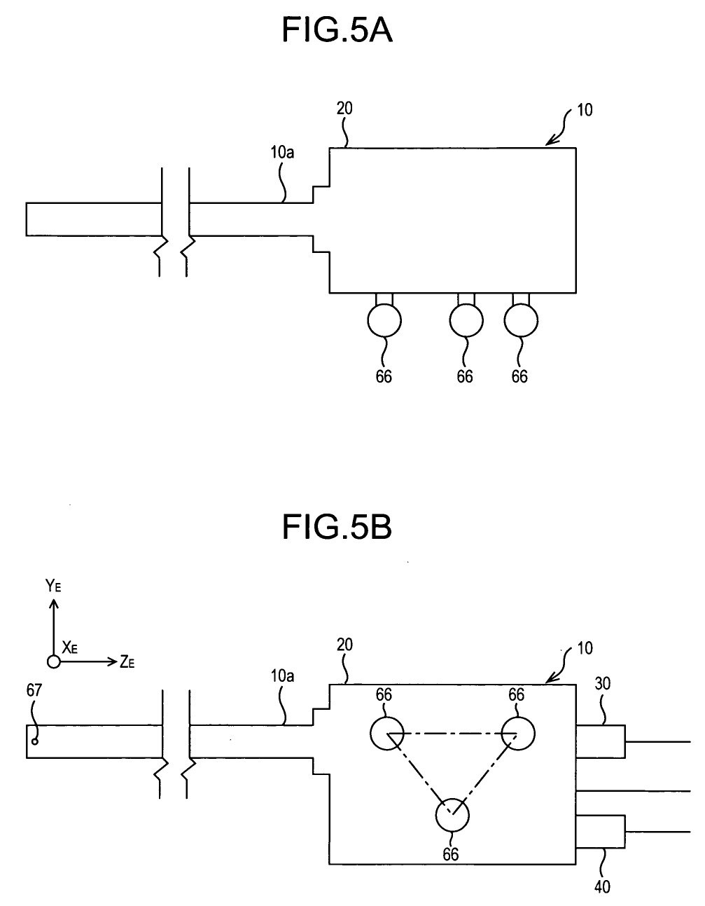

Next, a mechanism to measure the position of the endoscope device 10 will be described. Three endoscope markers 66 are attached to the side surface of the image separating device 20 of the endoscope device 10 as shown in FIG. 5A and FIG. 5B. Each of the endoscope markers 66 is a spherical shaped marker. Retroreflection sheet is pasted on the surface of each marker. The three-dimensional position of the endoscope device 10 can be specified by measuring the markers 66 by the position measuring device 70.

The local coordinate system of the endoscope device 10 is defined as shown in FIG. 5B.

Local Coordinate System of the Endoscope Device: (X_{E}, Y_{E}, Z_{E})

An origin 67 of the local coordinate system of the endoscope device 10 is a pupil position of the rigid endoscope 10a. And the axes X_{E} and Y_{E} are parallel to the axes X_{S} and Y_{S}, respectively.

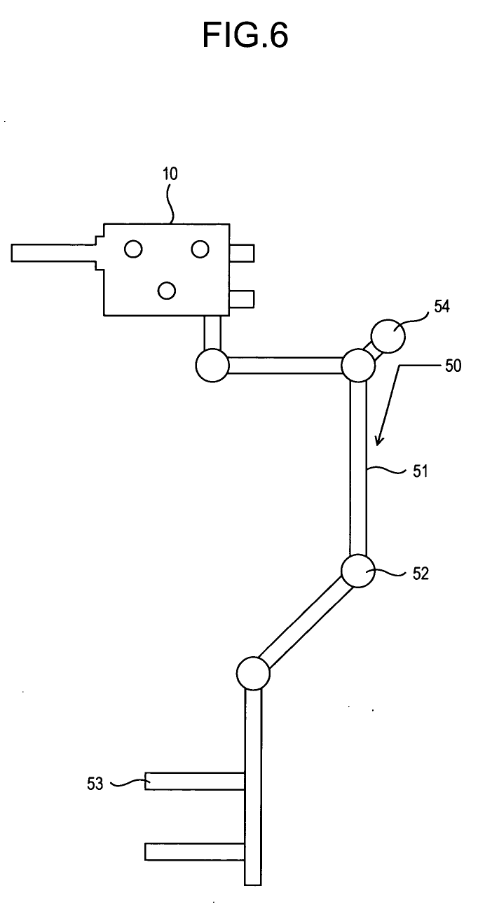

The endoscope holding mechanism 50 that holds the endoscope device 10 consists of links 51, joints 52 and a fixing portion 53 to form an arm-like shape as shown in FIG. 6. The endoscope holding mechanism 50 has a connecting portion (not shown) that is connected to the endoscope device 10, and the endoscope device 10 is detachable and attachable to the holding mechanism 50. Further, the endoscope holding mechanism 50 can be fixed to a bed in an operating room using the fixing portion 53. When the endoscope device 10 is used, an operator attaches the endoscope device 10 to the holding mechanism 50. Next, the operator fixes the holding mechanism 50 with the endoscope device 10 to the bed. Then, the operator moves the endoscope device 10 to a desired position.

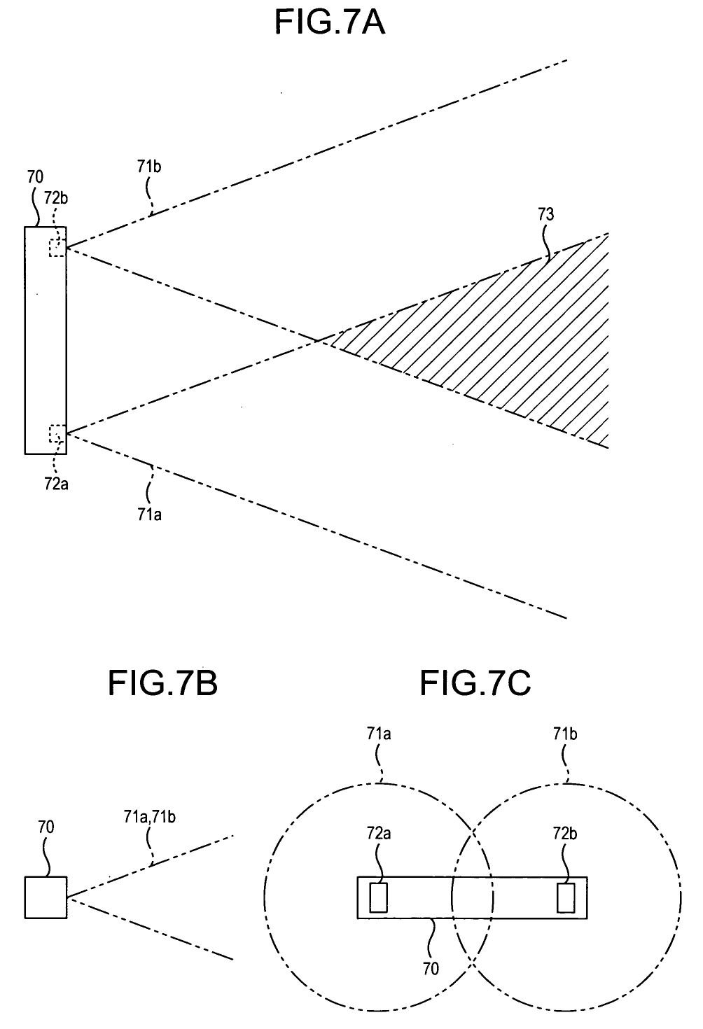

The position measuring device 70 measures the positions of the endoscope device 10 and the perspective image reference position marker 90. Specifically, POLARIS (Northern Digital Inc.) can be used as the position measuring device 70. As shown in FIG. 7A and FIG. 7C, the body of the position measuring device 70 has a rectangular parallelepiped shape, and a pair of light emitting/receiving portions 72a and 72b are mounted at both ends of the body. Each of the light emitting/receiving portions 72a and 72b emits infrared light and receives infrared light reflected from the endoscope markers 66 attached to the endoscope device 10 and from the perspective image reference position marker 90. The position measuring device 70 measures the three-dimensional positions of the endoscope device 10 and the perspective image reference position marker 90 based on the condition of the received infrared light. References 71a and 71b represent measurement areas of the light emitting/receiving portions 72a and 72b, respectively, and the three-dimensional position can be measured when an object is located within the overlapped area 73.

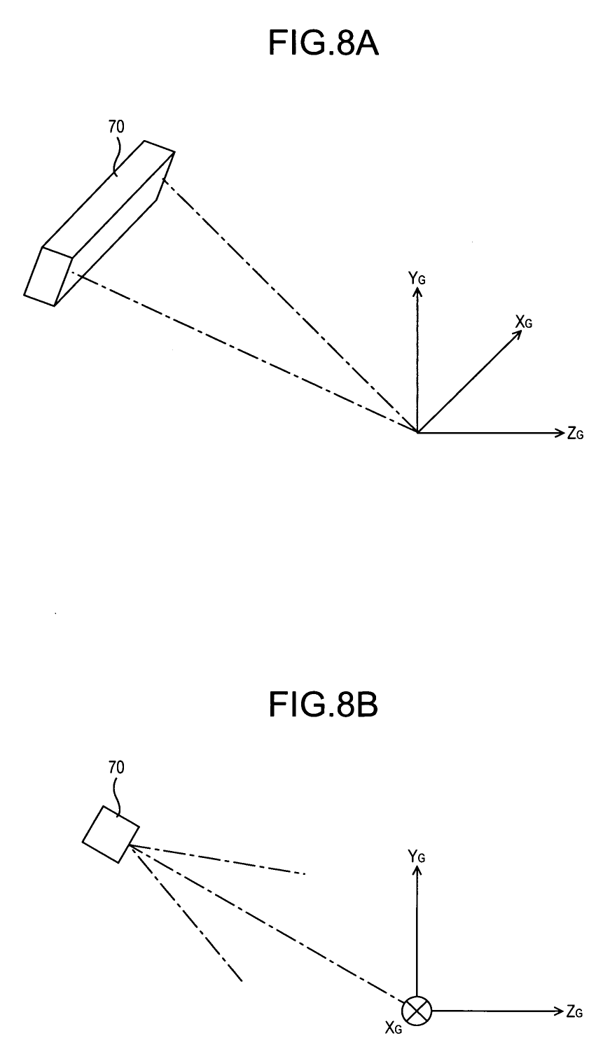

The three-dimensional coordinate system measured by the position measuring device 70 becomes a reference coordinate system when the perspective image is composed to the endoscopic image, and it is defined as shown in FIG. 8A and FIG. 8B.

Coordinate System of the Position Measuring Device: (X_{G}, Y_{G}, Z_{G})

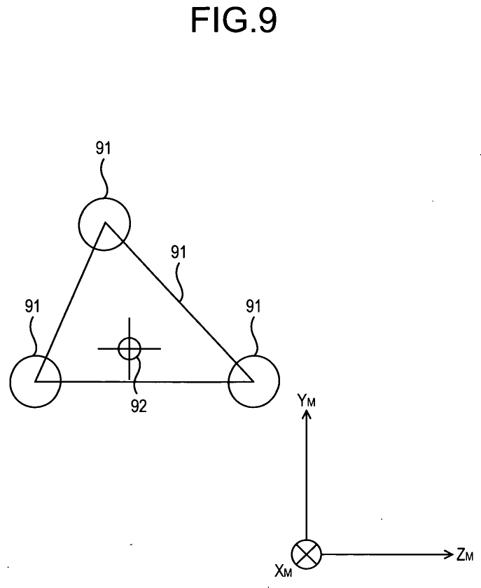

In addition, the perspective image reference position marker 90 defines a reference point when the coordinate system of the perspective image and the coordinate system of the endoscope are composed. The perspective image reference position marker 90 includes three reflecting spheres 91 as shown in FIG. 9. Retroreflection sheet is pasted on the surface of each reflecting sphere 91. The position measuring device 70 measures the three-dimensional position of the perspective image reference position marker 90 by receiving the infrared light reflected from the respective reflecting spheres 91. The material of the marker needs to be taken by a CT scanner and to have X-lay shield factor that is the same as or larger than that of a human bone. A reference 92 is an origin of the coordinate system of the perspective image reference position marker 90.

The coordinate system of the perspective image reference position marker is defined as shown in FIG. 9.

Coordinate System of the Perspective Image Reference Position Marker: (X_{M}, Y_{M}, Z_{M})

When sectional images are captured by the tomography scanner 100, the perspective image reference position marker 90 is fixed to a body surface of a patient as a subject. The tomography scanner 100 captures the sectional images of the patient with the perspective image reference position marker 90. The perspective images are created based on the sectional images. The position of the perspective image reference position marker 90 is a reference point in the perspective image when the perspective image is composed to the endoscopic image.

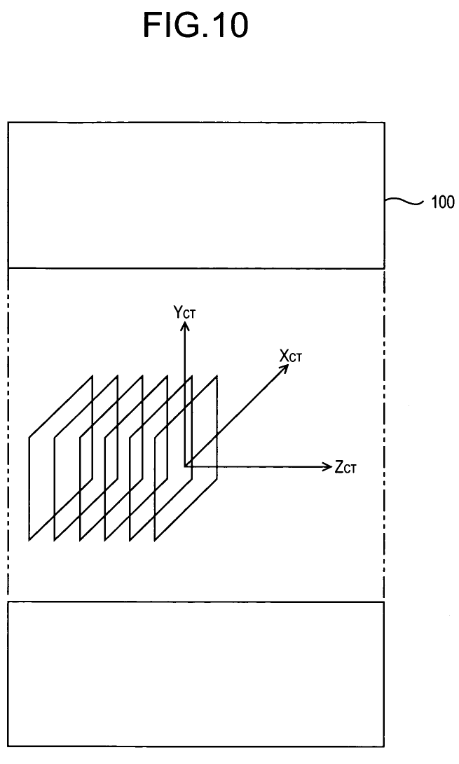

The tomography scanner 100 is a CT scanner or an MRI machine or the like, and the coordinate system thereof is defined as shown in FIG. 10.

Coordinate System of the Tomography Scanner: (X_{CT}, Y_{CT}, Z_{CT})

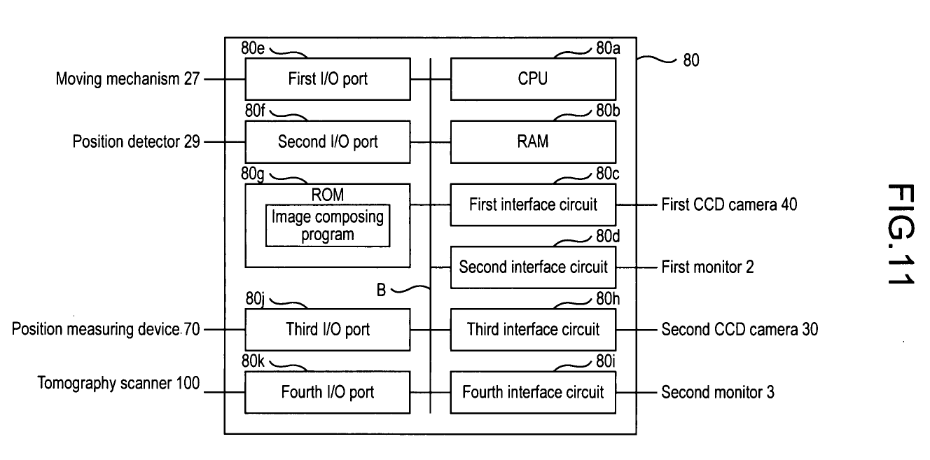

The image composing device 80 composes the endoscopic image that is taken by the endoscope device 10 with the perspective image created based on the sectional images that are captured by the tomography scanner 100. As shown in FIG. 11, the image composing device 80 is provided with a CPU 80a, a RAM 80b, a first interface circuit 80c, a second interface circuit 80d, a first I/O port 80e, a second I/O port 80f, a third I/O port 80j, a fourth I/O port 80k, a third interface circuit 80h and a fourth interface circuit 80i.

The CPU 80a is a central processing unit that totally controls the respective hardware devices 80b through 80k. The RAM 80b is a random access memory that cashes various programs read by the CPU 80a and on which a working area of the CPU 80a is developed. The ROM 80g stores data and various programs including an image composing program.

The first interface circuit 80c is responsible for receiving the image signal from the first CCD camera 40. The second interface circuit 80d is responsible for transmitting the image signal to the first monitor 2. Receiving an instruction from the CPU 80a, the first I/O port 80e receives the information (the X stage moving amount information and the Y stage moving amount information) representing the moving amounts of the X stage and the Y stage (that is, the shifting amount of the optical axis Ax) from the moving mechanism 27. The second I/O port 80f receives the zoom position information from the position detector 29 according to an instruction from the CPU 80a. The third I/O port 80j receives the position information of the endoscope device 10 and that of the perspective image reference position marker 90 from the position measuring device 70 according to an instruction from the CPU 80a. The fourth I/O port 80k receives the three-dimensional image information from the tomography scanner 100 according to an instruction from the CPU 80a. The third interface circuit 80h is responsible for receiving the image signal from the second CCD camera 30. The fourth interface circuit 80i is responsible for transmitting the image signal to the second monitor 3.

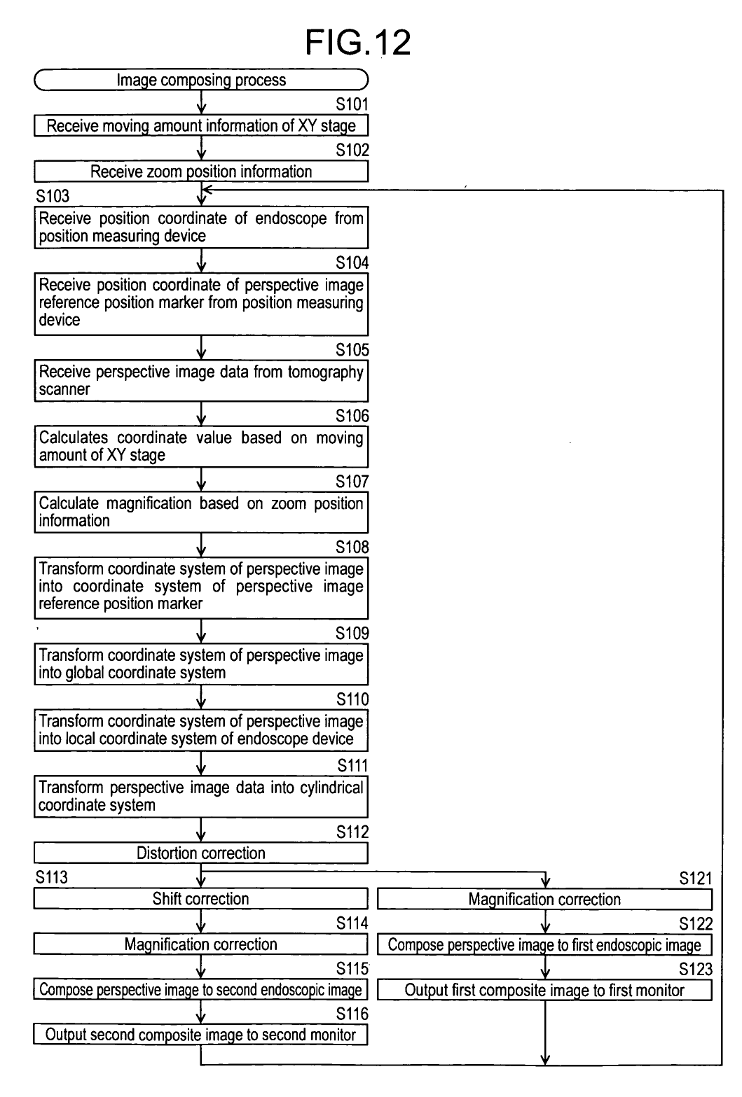

Next, the image composing process will be described with reference to the flowchart in FIG. 12. The image composing process starts at the time when the CPU 80a receives the image signal from the first CCD camera 40 through the first interface circuit 80c. Starting the process, the CPU 80a receives the X stage moving amount information and the Y stage moving amount information from the moving mechanism 27 through the first I/O port 80e (S101).

Then, the CPU 80a receives the zoom position information from the position detector 29 through the second I/O port 80f (S102), receives the position coordinate of the endoscope device 10 from the position measuring device 70 through the third I/O port 80j (S103), receives the position coordinate of the perspective image reference position marker 90 from the position measuring device 70 through the third I/O port 80j (S104), and receives the perspective image data from the tomography scanner 100 through the fourth I/O port 80k (S105).

Next, the CPU 80a updates the position information of the respective stages stored in the RAM 80b based on the X-stage moving amount information and the Y-stage moving amount information, and calculates the coordinate value representing the position of the optical axis Bx of the second re-imaging lens 26 in the plane coordinate, which defines the image area displayed based on the image signal created by the first CCD camera 40, according to the X-stage moving amount information and the Y-stage moving amount information (S106). It is because the position of the optical axis Bx of the second re-imaging lens 26 depends on the position of the Pechan prism 24 moved by the XY stage 27a in the XY plane.

Next, the CPU 80a calculates the magnification of the second re-imaging lens 26 based on the zoom position information (S107).

Then, the CPU 80a transforms the coordinate system of the perspective image from the coordinate system of the tomography scanner 100 to that of the perspective image reference position marker (S108). That is, the tomography scanner 100 captures sectional images of the patient and the perspective image reference position marker 90 at the same time. The CPU 80a measures a deviation amount and a rotation angle between the coordinate system of the tomography scanner 100 and the coordinate system of the perspective image reference position marker 90 based on the captured images and performs the coordinate transformation according to the measurement values.

Next, the CPU 80a transforms the perspective image data transformed in the coordinate system of the perspective image reference position marker into the coordinate system of the position measuring device 70 (a global coordinate system) (S109). That is, the origin position and the rotation data of the perspective image reference position marker 90 are detected by the position measuring device 70 and the coordinate transformation of the perspective image data is performed based on the detected values.

Next, the CPU 80a transforms the perspective image data transformed in the global coordinate system into the local coordinate system of the endoscope device (S110). That is, the perspective image data is transformed into the local coordinate system of the endoscope (X_{E}, Y_{E}, Z_{E}) according to the origin position, the rotation data of the perspective image reference position marker 90, and the origin position, the rotation data of the endoscope device 10 that are detected by the position measuring device 70.

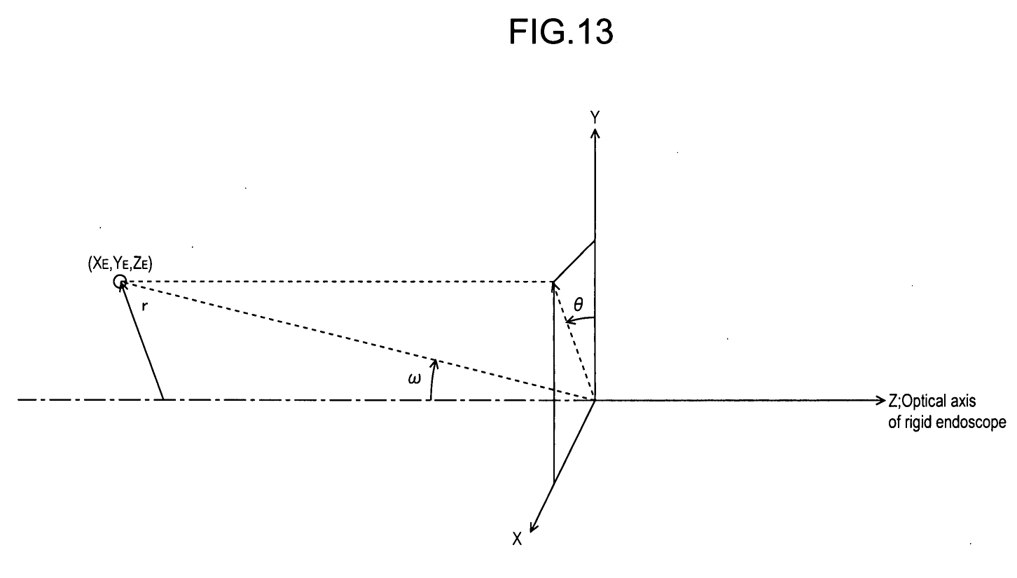

Next, the CPU 80a transforms the perspective image data (X_{E}, Y_{E}, Z_{E}) into the cylindrical coordinate system (r, θ, Z_{E}) to correct an effect of distortion of the rigid endoscope 10a (S111). That is, as shown in FIG. 13, the local coordinate of the endoscope (rectangular coordinates) is transformed into the cylindrical coordinate system.
r=\sqrt{X_{E}²+Y_{E}²}
r=Z_{E}·tan ω
X_{E}=r·cos θ
Y_{E}=r·sin θ

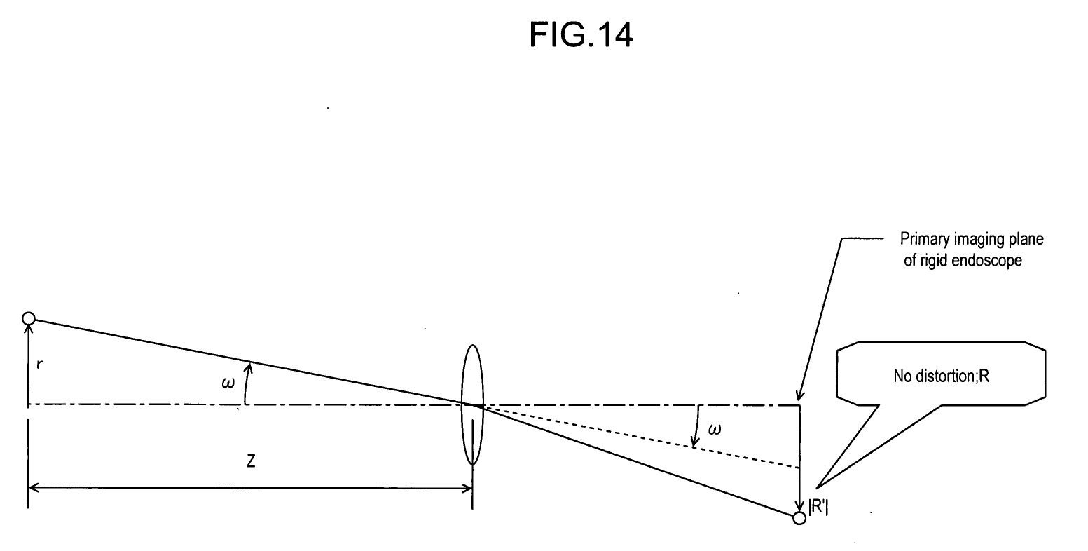

Next, the distortion DIST included in the perspective image transformed into the cylindrical coordinate system is corrected (S112). As shown in FIG. 14, an image height of an object whose height is r on the primary imaging plane of the rigid endoscope becomes R when there is no distortion or R′ when there is distortion. The image height R′ is represented by the image height R as follows.
R′=Rξ3·R³+ξ5·R⁵+ξ7·R⁷+ . . .
Where, R=f·tan ω=f·r/Z,
f is focal length of the rigid endoscope, Z is an object distance and
   DIST=ξ3·R²+ξ5R⁴+ξ7·R⁶+ . . .

Next, the image data on the primary imaging plane of the rigid endoscope in the polar coordinate system is transformed into a rectangular coordinate system. Defining the rectangular coordinate on the imaging plane of the rigid endoscope as (X_{E}′, Y_{E}′), the coordinate can be transformed as follows.
X_{E}′=R′·cos θ
Y_{E}′=R′·sin θ

Then, the CPU 80a transforms the perspective image using the coordinate system transformed as described above and composes it with the first endoscopic image taken by the first CCD camera 40 and with the second endoscopic image taken by the second CCD camera 30. These processes are executed by parallel processing when the image composing device 80 has a plurality of CPUs or are executed by time sharing processing when the device 80 has a single CPU.

The perspective image that will be composed with the first endoscopic image is corrected so that its magnification is identical to that of the first endoscopic image (S121). Assuming that the optical magnification of the first image taking optical system is m₁, the perspective image transformed in the coordinate system of the primary imaging plane of the rigid endoscope should be multiplied by the magnification m₁ so that the size of the perspective image is identical to that of the first endoscopic image. The coordinate (T, U) on the first CCD camera 40 is defined as follows.
T=m₁·X_{E}′
U=m₁·Y_{E}′

Next, the perspective image after the magnification correction is composed with the first endoscopic image (S122), the first endoscopic image over which the perspective image is superimposed is output from the second interface circuit 80d to display the first composite image on the first monitor 2 (S123).

On the other hand, the perspective image that will be composed with the second endoscopic image is corrected to shift the display area corresponding to the change of the image taking area of the endoscopic image taken by the second image taking optical system associated with the shift by the first shifting mechanism (S113). The process of the shift correction (S113) corresponds to the second shifting mechanism in the claims. The shift correction is executed based on the information (X stage moving amount information=ΔX_{S}, Y stage moving amount information=ΔY_{S}) representing the moving amounts of the X stage and the Y stage (that is, the shifting amount of the optical axis Ax) received from the first I/O port 80e. Since the shifting amount of the optical axis of the second image taking optical system is equal to twice the moving amount of the prism, the coordinate (X_{E}″, Y_{E}″) of the perspective image after the shift correction is represented as follows.
X_{E}″=X_{E}′−ΔX_{S}×2
Y_{E}″=Y_{E}′−ΔY_{S}×2

Next, the magnification of the perspective image is converted so that the magnification of the perspective image after the shift correction will be identical to that of the second endoscopic image (S114). The CPU 80a calculates the optical magnification m₂ of the second image taking optical system based on the zoom position information received from the position detector 29 through the second I/O port 80f. Then, the CPU 80a multiplies the perspective image after the shift correction by the magnification m₂ so that the magnification of the perspective image is identical to that of the second endoscopic image. The coordinate (V, W) on the second CCD camera 30 is represented as follows.
V=m₂·X_{E}″
W=m₂·Y_{E}″

Next, the CPU 80a composes the perspective image after the magnification correction to the second endoscopic image (S115) and outputs the second endoscopic image over which the perspective image is superimposed through the fourth interface circuit 80i to display the second composite image on the second monitor 3 (S116).

The processes from the step S103 to the step S123 are repeatedly executed, thereby the position of the endoscope device 10 is detected in real time and the wide and enlarged moving images that are composed of the endoscopic images and the internal structure captured by the tomography scanner such as a CT scanner based on the detected position on the first and second monitors 2 and 3, respectively.

When the diagnosis supporting device of the embodiment is operated, the endoscope device 10 is fixed near a patient by the holding mechanism 50. Then, the endoscopic image taken by the first image taking optical system including the objective optical system of the rigid endoscope 10a in the endoscope device 10 is composed to the perspective image in the corresponding area and the composed (superimposed) image is displayed on the first monitor 2. At the same time, the endoscopic image taken by the second image taking optical system including the objective optical system is composed to the perspective image in the corresponding area and the composed (superimposed) image is displayed on the second monitor 3.

Further, when the image taking area of the second image taking optical system is changed by moving the Pechan prism 24, the display area of the perspective image is changed in response to the moving amount. Therefore, the areas of the both images that are superimposed can be coincident with each other. During normal operation, the position measuring device 70 measures the relative position between the endoscope device 10 and the patient, and the image composing device 80 calculates the positional relationship between the endoscopic image and the perspective image based on the measured relative position to superimpose the images. In addition, since the image taking area of the second image taking optical system can be moved by moving the Pechan prism with staying the endoscope device 10 at the fixed position, if the path of the infrared light emitted from the position measuring device 70 to the endoscope device 10 is temporarily blocked by an operator or another devices (that is, if the position of the endoscope device 10 cannot be measured), the image taking area can be changed and the display area of the perspective image can be also changed correspondingly, which enables the operation without interruption, lightening load on a patient by reducing the operation time.

Still further, since the image composing process of the embodiment continuously detects the position of the endoscope device 10, the coordinate transformation and the image composition can be executed even if a patient involuntarily moves or the endoscope device is moved as an operation proceeds.

Hereinafter, the respective coordinate systems are listed.
(X_{E}, Y_{E}, Z_{E}): The local coordinate system of the endoscope device 10.
(X_{S}, Y_{S}): The coordinate system of the viewing field shifting mechanism.
(X_{G}, Y_{G}, Z_{G}): The coordinate system of the position measuring device (the global coordinate system).
(X_{M}, Y_{M}, Z_{M}): The coordinate system of the perspective image reference position marker.
(X_{CT}, Y_{CT}, Z_{CT}): The coordinate system of the tomography scanner.
(r, θ, Z_{E}): The local cylindrical coordinate system of the endoscope device 10.
(R′, θ): The local cylindrical coordinate system on the primary imaging plane of the endoscope device 10.
(X_{E}′, Y_{E}′): The local rectangular coordinate system on the primary imaging plane of the endoscope device 10.
(T, U): The coordinate system on the first image sensor of the endoscope device 10.
(V, W): The coordinate system on the second image sensor of the endoscope device 10.

The present disclosure relates to the subject matter contained in Japanese Patent Application No. P2003-424646, filed on Dec. 22, 2003, which are expressly incorporated herein by reference in its entirety.

1. A diagnosis supporting device, comprising:
an endoscope device that takes an image of an internal structure of a subject by forming said image on an image sensor through an optical system;
a holding mechanism that holds said endoscope device so that said endoscope device can be fixed with respect to said subject;
an image composing device that superimposes a perspective image of a predetermined area of said subject that is created based on sectional images obtained by a tomography scanner over an endoscopic image of said predetermined area taken by said endoscope device;
a displaying device that displays the image composed by said image composing device;
a first shifting mechanism that relatively shifts the position of said image formed by said optical system of said endoscope device and the position of said image sensor; and
a second shifting mechanism that shifts the display area of said perspective image corresponding to the change of the image taking area by said first shifting mechanism,
wherein the endoscopic image shifted by said first shifting mechanism and the perspective image shifted by said second shifting mechanism are composed by said image composing device, and the composed image is displayed on said displaying device.

2. The diagnosis supporting device according to claim 1, wherein said tomography scanner is a CT scanner or an MRI machine.

3. The diagnosis supporting device according to claim 1, wherein said first shifting mechanism comprises a Pechan prism included in said optical system of said endoscope device, and a prism moving mechanism that moves said Pechan prism in two-dimensional direction in a plane perpendicular to the optical axis.

4. The diagnosis supporting device according to claim 1, wherein said endoscope device comprises:
an objective optical system that forms an image of said subject;
a first re-imaging optical system that re-images a predetermined area of said image formed by said objective optical system;
a first image sensor that takes the image formed by said first re-imaging optical system;
a second re-imaging optical system that enlarges and re-images a part of said predetermined area of said image formed by said objective optical system; and
a second image sensor that takes the image formed by said second re-imaging optical system,
wherein said image composing device creates a first composite image by composing a first endoscopic image taken by said first image sensor with a perspective image of the corresponding area and creates a second composite image by composing a second endoscopic image taken by said second image sensor with a perspective image of the corresponding area,
wherein said first shifting mechanism shifts the relative positions of the image formed by said second re-imaging optical system and said second image sensor, and said second shifting mechanism shifts the perspective image that will constitute said second composite image, and
wherein said displaying device comprises a first monitor for displaying said first composite image and a second monitor for displaying said second composite image.

5. A diagnosis supporting device, comprising:
a position measuring device for measuring a reference position of an endoscope device as a first coordinate value and a reference position of a perspective image as a second coordinate value when said endoscope device is set up;
a first image taking optical system;
a first image sensor that takes an image within a predetermined area in a view field through said first image taking optical system and that outputs a first image signal;
a second image taking optical system that includes at least one lens and forms an image within at least a part of said predetermined area;
a second image sensor that takes an image through said second image taking optical system and that outputs a second image signal;
a shifting device that moves the image taking area of said second image sensor through said second image taking optical system within said predetermined area by relatively shifting the optical axis of a lens in said second image taking optical system with respect to said second image sensor;
an endoscope device that outputs the shift amount between the optical axis of said lens that is shifted by said shifting device and said second image sensor as a third coordinate value;
an image composing device that composes said first image signal and said perspective image based on said first and second coordinate values and composes said second image signal and said perspective image based on said first, second and third coordinate values;
a first monitor that displays said first image signal output from said image composing device; and
a second monitor that displays said second image signal output from said image composing device.
